(19) United States
(12) Plant Patent Application Publication
(10) Pub. No.: US 2014/0033378 P1
(43) Pub. Date: Jan. 30, 2014

(54) Grapevine 'IFG Thirteen'

(50) ;

(71) Applicant: David Cain, Bakersfield, CA (US)

(72) Inventor: David Cain, Bakersfield, CA (US)

(73) Assignee: International Fruit Genetics, LLC, Bakersfield, CA (US)

(21) Appl. No.: 13/507,753

(22) Filed: Jul. 26, 2012

(51) Int. Cl. A01H 5/00

(52) U.S. Cl. PLT/205

(57) ABSTRACT

This invention is a new and distinct grapevine variety denominated ‘IFG Thirteen’. The new grapevine is characterized by producing obtuse ovate, completely black seedless berries which are firm in texture, low in acidity and ripen early in the growing season.

### LATIN NAME OF THE GENUS AND SPECIES CLAIMED

Vitis vinifera

### VARIETY DENOMINATION

‘IFG Thirteen’

### BACKGROUND OF THE INVENTION

The new and distinct grapevine described and claimed herein originated from a hand pollinated cross of the IS 283 variety (non-patented) and the Fantasy variety (non-patented) hybridized in May 2001. The abortive seed traces were subsequently embryo cultured and the resulting population of plants were planted in the field in April 2002. The present variety of grapevine was selected as a single plant in August 2003 and was first asexually propagated by hardwood cuttings in December 2003. The resulting propagules were planted during April 2004 near Delano, Kern County, Calif. and were found to reproduce true-to-type through at least three generations of asexual reproduction.

### BRIEF SUMMARY OF THE INVENTION

The new grapevine ‘IFG Thirteen’ is characterized by producing obtuse ovate, completely black seedless berries which are firm in texture, low in acidity and ripen early in the growing season. Fruits normally ripen in late July to early August near Delano, Calif.

To the inventor's knowledge, the known variety which the new grapevine variety is most similar to is the ‘IFG One’ variety. ‘IFG Thirteen’ can be distinguished from the ‘IFG One’ variety by its producing slightly firmer, smaller berries (about 5.5 grams as opposed to about 6.1 grams for ‘IFG One’) that are slightly lower in acidity and slightly more obtuse in shape and are less prone to tip cracking and berry shatter. The ‘IFG Thirteen’ stores better than the ‘IFG One’ variety.

The ‘IFG Thirteen’ variety can be distinguished maternal parent the ‘IS 283’ variety by ripening slightly later, having firmer flesh, exhibiting less variability in berry size and having smaller residual seed traces.

The ‘IFG Thirteen’ variety can be distinguished from its paternal parent the Fantasy variety by producing smaller, less elongated, earlier ripening fruits that are less prone to splitting.

### BRIEF DESCRIPTION OF THE FIGURE

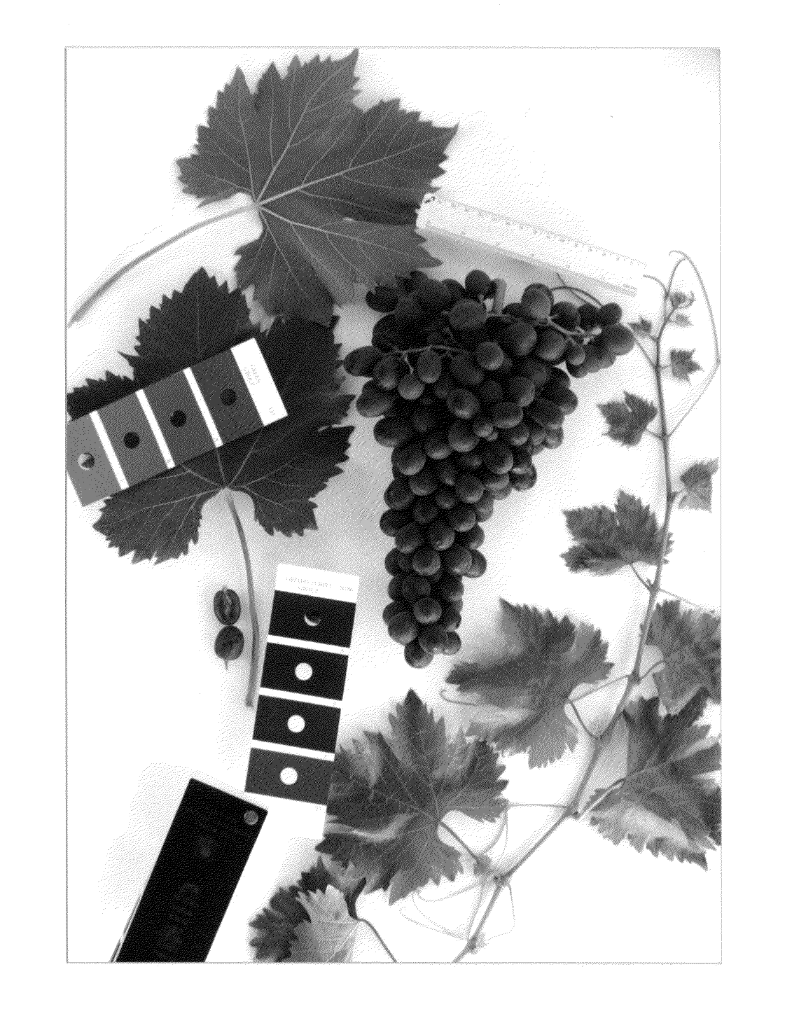

The accompanying photographic illustration in FIG. 1 illustrates in full color ‘IFG Thirteen’. The photograph was taken outdoors with indirect lighting. The colors are as nearly true as is reasonably possible in a color representation of this type.

### DETAILED BOTANICAL DESCRIPTION OF THE INVENTION

Throughout this specification, color names beginning with a small letter signify that the name of that color, as used in common speech, is aptly descriptive. Color names beginning with a capital letter designate values based upon R.H.S. Colour Chart, published by The Royal Horticultural Society, London, England.

Throughout this specification subjective description values conform to those set forth by the International Plant Genetic Resources Institute publication ‘Descriptors for Grape’ (vitis spp.) (1983) which was developed in collaboration with the Office International de la Vigne et du Vin (OIV) and the International Union for the Protection of New Varieties of Plants (UPOV).

The descriptive matter which follows pertains to ‘IFG Thirteen’ plants grown in the vicinity of Delano, Kern County, Calif. during 2009 and 2010, and is believed to apply to plants of the variety grown under similar conditions of soil and climate elsewhere:
Vine:
   General.—Size — Large Vigor — Vigorous Density of foliage — Dense Productivity — Productive Root stock — Own root Training method — Typically spur pruned leaving 2 bud spurs.
   Trunk.—Trunk diameter of 7-year-old vines at 30 cm above the soil line — 9.2 cm Shape — Stocky Straps — Long — split Surface texture — Shaggy Inner bark color — Greyed-orange 165A.
Shoots:
   Young Shoot.—Form of tip — Fully opened Distribution of anthocyanin coloration of tip — Piping (striped) Intensity of anthocyanin coloration of tip — Weak Density of prostrate hairs of tip — Sparse — Medium Density of erect hairs of tip — Absent Color — 146A, B.
   Woody shoot (Mature canes).—Shape — Medium Internode length — Medium; About 12.4 cm Width at node — About 1.2 cm Cross section — Circular Surface — Striate to slightly ribbed Main color — Greyed orange; 165A, B, 164A, B Density of erect hairs of nodes — None Density of erect hairs on internodes — None Growth of axillary shoots — Medium; Approximately 12.7 cm.
   Flowering shoot.—Vigor during flowering — Strong Attitude during flowering on shoots not tied — Semi-erect Color — dorsal side of internodes — Green with Red stripes Color — ventral side of internodes — Green Color — dorsal side of nodes — Green with Red stripes Color — ventral side of nodes — Green Density of prostrate hairs on nodes — Sparse Density of erect hairs on nodes — None Density of prostrate hairs on internode — None Density of erect hairs on internode — None Anthocyanin coloration of buds — Present.
   Tendrils.—Distribution on the shoot (at full flowering) — Discontinuous Length of Tendril — Medium; About 19.9 cm Thickness — Medium Color — 152B, N144A Form — bifurcated Number of consecutive tendrils — 2.
Leaves:
   Young leaves.—Color of upper surface of first four distal unfolded leaves — Green Average intensity of anthocyanin coloration of six distal leaves prior to flowering — Weak Density of prostrate hairs between veins (lower surface) — Very sparse Density of prostrate hairs on veins (lower surface) — Sparse Density of erect hairs between veins (lower surface) — Absent Density of erect hairs on veins (lower surface) — Very sparse.
   Mature leaves.—Average length — About 16.4 cm Average width — About 19.7 cm Mature leaf size — Large Shape of blade — Pentagonal Number of lobes — 5 Anthocyanin coloration of main veins on upper side of blade — Very weak to weak Mature leaf profile — Undulate Blistering surface of blade upper surface — Weak Leaf blade tip — Curved downwardly slightly Undulation of margin — Medium Thickness — Medium Undulation of blade between main and lateral veins — Overall Shape of teeth — Mixture of both sides straight and both sides convex Length of teeth — Medium — long Ratio length/width of teeth — Equal Shape of upper lateral sinuses — Lobes slightly overlapping Depth of upper lateral sinuses — Medium General shape petiole sinus — Half open Shape of base of upper leaf sinuses — V-shaped Tooth at petiole sinus — Absent Density of prostrate hairs between veins on lower surface of blade — Absent Density if erect hairs between veins on lower surface of blade — Absent Density of prostrate hairs on main veins on lower surface of blade — very sparse Density of erect hairs on main veins on lower surface of blade — Sparse Density of prostrate hairs on main veins on upper surface of blade — very sparse Density of erect hairs on main veins on upper surface of blade — None Autumn coloration of leaves — Green; 139A, Greyed-purple; N186A, B, 187A, B.
   Upper surface.—Color — 138A Anthocyanin coloration of main veins — Very weak to weak Surface appearance — Dull Blistering surface of blade — Weak.
   Lower surface.—Color — 138A, B, C Anthocyanin coloration of main veins (lower surface) — Weak Glossiness — Medium Surface texture — Smooth Surface appearance — Semi-glossy.
   Petiole.—Length — About 15.6 cm Length of petiole compared to middle vein — Slightly longer Density of prostrate hairs on petiole — None Density of erect hairs on petiole — Sparse.
   Buds.—Bud fruitfulness — Basal: Mostly fruitful Position of first fruitful shoot on previous season cane — 1^{st}-2^{nd} node Time of bud burst — Early; Mar. 14, 2012.
Flowers:
   General.—Flower sex — Hermaphrodite Length of first inflorescence — Medium; About 24.2 mm long by 13.8 mm wide Position of first flowering and fruiting node — 4^{th} (current season growth) Number of inflorescence per flowering shoot — 1.1 to 2 Time of bloom — Early as compared with similar varieties in the growing area of Delano, Calif. Date of full bloom — May 13, 2012.
Fruit:
   General.—Ripening period — Early; Approximately; Aug. 16, 2011 Use — Fresh market Keeping quality — Good Resistance to — Insects: Average typical of Vitis vinifera species. Diseases: Average typical of Vitis vinifera species. Refractometer test — solid-sugar: About 15.6 Brix Brix/acid — About 40 Titratable acidity — About 0.39 Juice pH — About 3.92.
   Cluster.—Mature cluster length (peduncle excluded) — About 12.5 cm Mature cluster width — About 16.8 cm Mature cluster weight — About 1285 g Bunch density — Medium Number of berries — About 305 Form — Conical.
   Peduncle.—Lignification of peduncle — Weak Length of peduncle — Medium; Approximately 4.7 cm.
   Berry.—Uniformity of size — Uniform Single berry weight — About 5.5 g natural Shape — Obtuse ovate Seeds — Contains small rudimentary seed traces Cross section. — Circular Berry dimensions — Longitudinal axis: About 2.6 cm. Horizontal axis: About 1.9 cm Berry firmness — Medium firm Particular flavor — Neutral Bloom (cuticular wax) — Medium Berry separation from pedicel — Medium Skin color (without bloom) — Greyed-purple; N186A, 187A.
   Skin.—Thickness — Medium Texture. — Medium Reticulation — Absent Tenacity — Tenacious to flesh.

What is claimed:

1. A new and distinct variety of grapevine as herein illustrated and described.
